(19) United States
(12) Patent Application Publication
(10) Pub. No.: US 2010/0028962 A1
(43) Pub. Date: Feb. 4, 2010

(54) Algal Medium Chain Length Fatty Acids and Hydrocarbons

(76) Inventors: Qiang Hu, Chandler, AZ (US); Milton Summerfeld, Chandler, AZ (US)

Correspondence Address: MCANDREWS HELD & MALLOY, LTD, 500 WEST MADISON STREET, SUITE 3400, CHICAGO, IL, 60661, US

(21) Appl. No.: 12/441,233

(22) PCT Filed: Sep. 18, 2007
(86) PCT No.: PCT/US2007/078760

§ 371 Date: Aug. 28, 2009

Related U.S. Application Data
(60) Provisional application No. 60/825,946, filed on Sep. 18, 2006.

(51) Int. Cl. C12P 7/64; C12N 1/12; C12P 5/00; C07C 11/00

(52) U.S. Cl. 435/134; 435/257.1; 435/166; 585/16

(57) ABSTRACT

The present invention provides methods and compositions for production of algal-based medium chain fatty acids and hydrocarbons.

### CROSS REFERENCE

This application claims priority to U.S. Provisional Patent Application Ser. No. 60/825946 filed Sep. 18, 2006, incorporated by reference herein in its entirety.

### BACKGROUND OF THE INVENTION

JP-8 is a kerosene-type military jet fuel derived from petroleum and is being used as the primary fuel for land-based air and ground forces (e.g., aircraft, ground vehicles, and equipment). The US Department of Defense (DOD) is the single largest oil consuming government body in the US, consuming over 90 million barrels of JP-8 in fiscal 2006, which represents about 15% of kerosene-based jet fuel produced by the U.S.

Commercial jet fuel similar to JP-8 in chemical composition is largely consumed by the U.S. commercial (corporate/private) aviation industry with passenger and cargo carriers burning nearly 500 million barrels of jet fuel in 2005. As having already consumed over 80% of its proven oil reserves, the U.S. now imports more than 60% of its oil. It is anticipated that within 20 years the U.S. will be importing from 80% to 90% of its oil. Much of this imported oil is supplied from nations in politically-volatile regions of the world where political instability, human rights abuses, and terrorism are the constant threat to a stable oil supply for the U.S. Over $250 billion is spent on foreign oil annually, representing a third of the growing US trade deficit and an increasing burden on the US economy. Although the U.S. can continue to increasingly import foreign oil, global oil supplies are not infinite. Even based upon an optimistic estimate of the world oil resource of approximately 2,200-3,900 billion barrels, nearly twice the proven reserve, the world supply of petroleum oil will be depleted within 40 years. Demand for oil by emerging and rapidly growing economies in China, India, and elsewhere, is also increasing competition and price volatility for limited global supplies. The severity of potential impacts of oil reduction on U.S. military operations, national security, and the growing economy will depend on how much, how quickly, and how far in advance of this event we are able to provide a wide range of renewable, affordable alternatives to JP-8 and other fossil fuels.

Oil-rich crops and algae are widely regarded as the most promising biological systems for cost-effective, sustainable production of biodiesel particularly for transportation. However, biodiesel produced from current available oil crop-based feedstocks and commercial processes is not suitable as a JP-8 surrogate fuel for military and commercial aviation applications due to its lower energy density and unacceptable cold-flow features. The disqualification of biodiesel as an alternative to JP-8 stems from the fact that the former contains mostly methyl esters of C16 and C18 fatty acids, whereas the latter has the main chemical components of C9 to C14 hydrocarbons. Compared to C9 to C14 hydrocarbons, oxygenated methyl esters of C 16 and C 18 fatty acids not only decrease energy density of the fuel, but also are responsible for high fuel viscosity, high flash point, and high freezing points (>−50° C.).

Biodiesel can be processed into JP-8 surrogate fuel through thermal, catalytic, and/or enzymatic processes. However, the subsequent secondary processing is neither cost-effective nor energy-efficient and consumes large quantities of fossil fuels with an energy conversion efficiency of 8% to 15%. This results in alternative jet fuel being prohibitively expensive and having unacceptably low energy efficiency. Clearly, transforming algae/plant-based oil or biodiesel into an affordable alternative to petroleum-derived JP-8 has great potential, but this will require significant innovations and improvements to current feedstock production systems and subsequent downstream processes to enhance oil conversion efficiency, while driving production costs down.

One way to increase energy conversion efficiency while reducing production costs of crop oil derived JP-8 surrogate fuel is to introduce certain feedstock oils that may naturally consist of large amounts of medium-chain fatty acids (C10 to C 14). The medium-chain fatty acids may require little cracking treatment, which is otherwise required process to break long-chain molecules into shorter ones. Coconut and palm kernel oils have turned out to be the exceptions from common oil crops by containing high concentrations (55˜69% of total fatty acids) of medium-chain (C12 and C14) fatty acids/esters. The world production of coconut oil was about 50 million metric tons in 1999, and the production of palm kernel oil was about 3.8 million tons in 2005. Indonesia, Malaysia, Philippines, and India are the major producers of coconut and palm kernel oils. These oils are mainly used for domestic consumption as food and cooking/frying oil. In the U.S. and other western countries, coconut and palm kernel oils are largely used in the manufacture of margarine and other fat/oil products, as well as in cosmetics, soaps, detergents and shampoos. Although coconut and palm kernel oils are being exploited for production of biodiesel and are considered to be kerosene-based jet fuel substitute, they are unlikely to be used as a major feedstock for jet fuel production due to limited supplies (Shay 1993; Srivastava & Prasad 2000).

An alternative is to make more medium-chain fatty acids through genetic manipulations of oil crops. However, the efforts made thus far with oil-crops have resulted in little commercial significance. This is due mainly to the lack of clear understanding of cellular/subcellular regulatory networks that may provide ‘global’ control over complex biochemical pathways, which may lead to partitioning of photosynthetically-fixed carbon specifically into the formation and accumulation of lipids/oil rather than biosynthesis of protein or carbohydrate. Lack of effective molecular genetic tools and methodologies is another major reason for unsuccessful strain improvement.

Microalgae may be a promising source of feedstock for biofuels because of a) their high lipid/oil contents (40 to 60% of dry weight); b) high specific growth rates (1 to 3 doubling time per day); c) the ability to thrive in saline/brackish water and utilize nutrients (N, P, and CO₂) from waste-streams (e.g., wastewater and flue gases from fossil fuel-fired power plants) for growth, and use marginal lands (desert, arid- and semi-arid lands) for wide-scale production all year around; and d) co-production of value-added products (e.g., biopolymers, proteins, polysaccharide, pigments). However, algal oils studied for biofuels so far are rather similar in chemical and physical properties to that of common crop oils, which are enriched with C16 to C18 fatty acids/esters.

### SUMMARY OF THE INVENTION

In a first aspect, the present invention provides methods for producing algal medium chain length fatty acids or hydrocarbons, comprising:
(a) culturing a first algal strain that can produce large quantities of a first medium chain length fatty acid subset, wherein the culturing is conducted under conditions suitable to promote production of the first medium chain fatty acid subset;
(b) culturing one or more further algal strains that can produce large quantities of a second or further medium chain length fatty acid subset, wherein the culturing is conducted under conditions suitable to promote production of the second medium chain fatty acid subset; and
(c) extracting oil from the first algal strain and the one or more further algal strains to produce a medium chain length combination; wherein the medium chain length combination comprises carbon chain length C10, C12, and C14 fatty acids or hydrocarbons, and wherein neither the oil from the first algal strain by itself nor the oil from any one of the one or more further algal strains by itself comprise detectable levels of each of carbon chain length C10, C12, and C14 fatty acids.

In a second aspect, the present invention provides methods for producing algal medium chain length fatty acids, comprising

(a) culturing Pinguiococcus pyrenoidosus under conditions suitable to promote production of medium chain length fatty acids; and

(b) extracting oil from the cultured Pinguiococcus pyrenoidosus, wherein the extracted oil comprises C14 and C16 chain length fatty acids.

In a third aspect, the present invention provides methods for producing algal medium chain length fatty acids or hydrocarbons, comprising

(a) culturing Pinguiococcus pyrenoidosus under conditions suitable to promote production of medium chain length fatty acids;

(b) culturing one or more further algal strains that can produce and accumulate large quantities of C10 and/or C12 chain length fatty acids, wherein the culturing is conducted under conditions suitable to promote production of the C10 and/or C12 chain length fatty acids; and

(c) extracting oil from the cultured Pinguiococcus pyrenoidosus and the one or more further algal strains to produce a medium chain length combination; wherein the medium chain length combination comprises carbon chain length C14 and one or more of carbon chain length C10 and C12 fatty acids or hydrocarbons.

In a fourth aspect, the present invention provides methods for producing algal medium chain length fatty acids or hydrocarbons, comprising

(a) culturing Trichodesmium erythraeum under conditions suitable to promote production of medium chain length fatty acids, wherein the medium chain length fatty acids comprise C10 chain length fatty acids;

(b) culturing Crypthecodinium sp. under conditions suitable to promote production of medium chain length fatty acids, wherein the medium chain length fatty acids comprise C12 chain length fatty acids; and

(c) extracting oil from the cultured Trichodesmium erythraeum and the cultured Crypthecodinium sp. to produce a medium chain length combination; wherein the medium chain length combination comprises carbon chain length C10 and C12 fatty acids or hydrocarbons.

In a fifth aspect, the present invention provides compositions comprising two or more isolated algal strains selected from the group consisting of Pinguiococcus pyrenoidosus, Aphanocapsa sp., Biddulphia aurita, Crypthecodinium sp., Emiliania huxleyi, Nitzschia alba, Prymnesium parvum, Skeletonema costatum, and Trichodesmium erythraeum, wherein the two or more algal strains make up at least 90% of the algae present in the composition.

In a sixth aspect, the present invention provides a substantially pure culture comprising
(a) growth medium; and
(b) the composition of any embodiment of the compositions of the fifth aspect of the invention.

In a seventh aspect, the present invention provides an algal-derived hydrocarbon fraction, produced by the methods of any embodiment of the first, second, third, or fourth aspects of the invention.

In an eighth aspect, the present invention provides an algal-derived, isolated medium chain hydrocarbon fraction, produced by the methods of any embodiment of the first, second, third, or fourth aspects of the invention.

In a ninth aspect, the present invention provides algal-derived kerosene produced by the methods of any embodiment of the first, second, third, or fourth aspects of the invention.

### BRIEF DESCRIPTION OF THE INVENTION

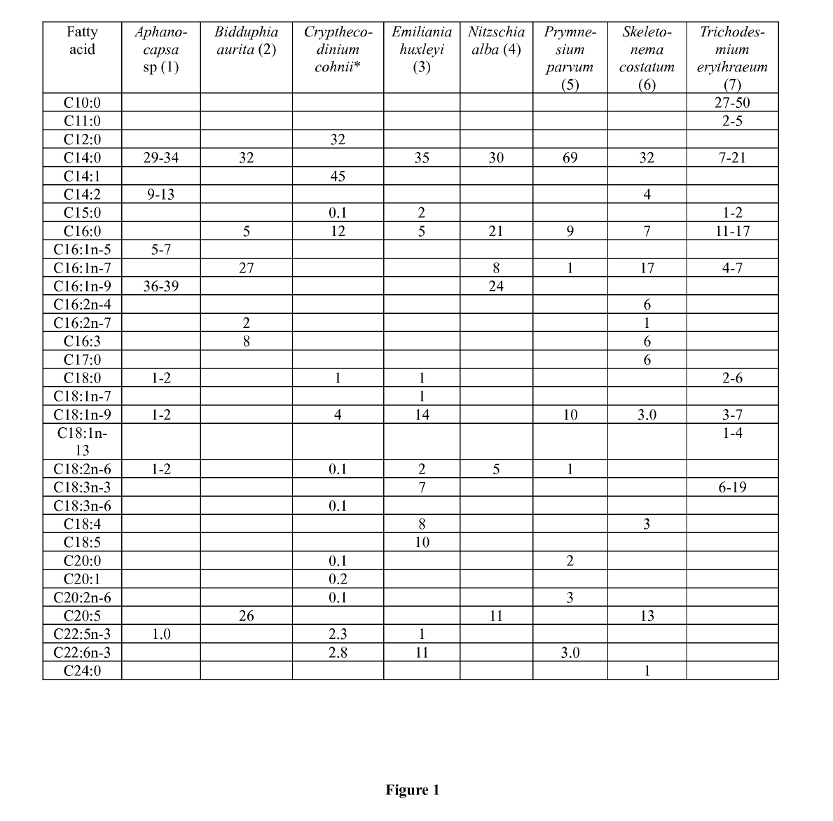

FIG. 1 shows data relating to total fatty acid content of representative algal strains for use in the present invention.

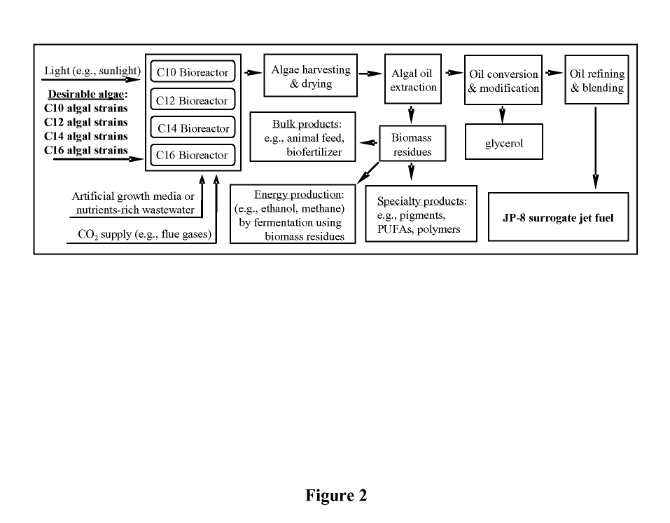

FIG. 2 is a flow-chart diagram of algae-based JP-8 surrogate jet fuel production.

### DETAILED DESCRIPTION OF THE INVENTION

In a first aspect, the present invention provides methods for producing algal medium chain length fatty acids or hydrocarbons, comprising:

(a) culturing a first algal strain that can produce large quantities of a first medium chain length fatty acid subset, wherein the culturing is conducted under conditions suitable to promote production of the first medium chain fatty acid subset;

(b) culturing one or more further algal strains that can produce large quantities of a second or further medium chain length fatty acid subset, wherein the culturing is conducted under conditions suitable to promote production of the second medium chain fatty acid subset; and

(c) extracting oil from the first algal strain and the one or more further algal strains to produce a medium chain length combination; wherein the medium chain length combination comprises carbon chain length C10, C12, and C14 fatty acids or hydrocarbons, and wherein neither the oil from the first algal strain by itself nor the oil from any one of the one or more further algal strains by itself comprise detectable levels of each of carbon chain length C10, C12, and C14 fatty acids.

Previous efforts to produce algal oil fractions enriched in medium chain length fatty acids used a cracking process to break long chain fatty acids/esters into shorter ones, followed by further processing. The methods of the present invention do not require such a cracking process, particularly when using algae that endogenously produce medium chain length fatty acids and not hydrocarbons. As a result, the methods of the invention allow isolation of algal fatty acids processing into a hydrocarbon fraction using, for example, a deoxygenation step. The methods of the invention can produce, for example, more kerosene-based jet fuel than “common” algal oils enriched with long chain fatty acids (C16 to C22) with a given amount of algal feedstock, and reduce capital and operational costs associated with the oil cracking and separation processes.

Algal oil enriched in medium chain length fatty acids can be used for various purposes, including but not limited to production of algal-based kerosene substitutes, high quality detergents, and research reagents (for example, isolated hydrocarbon fractions of a single chain length for use as standards that can be optionally labeled for research use).

As used herein, the phrase “medium chain length fatty acids” refers to fatty acids and esters thereof that range in carbon chain length from C8 to C16. In a further embodiment, medium chain length fatty acids range in carbon chain lengths from C9 to C14; in a further embodiment from C10 to C14. The two or more algal strains used (ie: 2, 3, 4, 5, or more algal strains) can produce and accumulate large quantities of medium chain length fatty acids. “Large quantities” means that 20% or more of total fatty acids produced by the algal strain are medium-chain length fatty acids. In a further embodiment, the two or more algal strains produce and accumulate at least 25% of the fatty acids produced as medium chain length fatty acids; more preferably, at least 30%, 35%, 40%, 45%, 50%, 55%, or more. Those of skill in the art will understand that while the algal strains employed produce medium-chain fatty acids, they may also produce other chain length fatty acids.

As used herein, the term “algae” or “algal strain” includes both microalgae and cyanobacteria. In one embodiment, the algae are eukaryotic microalgae. Non-limiting algal strains that can be used with the methods of the invention are provided in FIG. 1.

“Suitable conditions” for culturing algae are well known to those of skill in the art, and include appropriate light conditions (to promote photosynthetic growth), growth media (nutrients, pH, etc.), and CO₂ supply. The volume of growth medium can be any volume suitable for cultivation of the algae for methods of the invention. Any suitable nutrient supply can be used. Such nutrient supplies can include (or can supplemented by) wastewater or waste gases. In these embodiments, the methods further provide waste remediation benefits. For example, nutrient-contaminated water or wastewater (e.g., industrial wastewater, agricultural wastewater domestic wastewater, contaminated groundwater and surface water), or waste gases emitted from power generators burning natural gas or biogas, and flue gas emissions from fossil fuel fired power plants can be used as part of the growth medium. In these embodiments, the algae can be first cultivated in a primary growth medium, followed by addition of wastewater and/or waste gas. Alternatively, the algae can be cultivated solely in the wastestream source. When a particular nutrient or element is added into the culture medium, it will be taken up and assimilated by the algae. Typically, waste water is added to the culture medium at a desired rate. This water, being supplied from the waste water source, contains additional nutrients, such as phosphates, and/or trace elements (such as iron, zinc), which supplement the growth of the algae. In one embodiment, if the waste water being treated contains sufficient nutrients to sustain the microalgal growth, it may be possible to use less of the growth medium. As the waste water becomes cleaner due to algal treatment, the amount of growth medium can be increased. The major factors affecting waste-stream feeding rate include: 1) algal growth rate, 2) light intensity, 4) culture temperature, 5) initial nutrient concentrations in wastewater; 5) the specific uptake rate of certain nutrient/s; 6) design and performance of a specific bioreactor and 7) specific maintenance protocols.

Growth of the algae can be in any type of system or photobioreactor. As used herein, a “photobioreactor” is an industrial-scale culture vessel made of transparent clear materials (e.g., glass, acrylic, polycarbonate, PVC, etc) in which algae grow and proliferate. For use in this aspect of the invention, any type of system or photobioreactor can be used, including but not limited to open raceways (i.e. shallow ponds (water level ca. 15 to 30 cm high) each covering an area of 1000 to 5000 m₂ constructed as a loop in which the culture is circulated by a paddle-wheel (Richmond, 1986), closed systems, i.e. photobioreactors made of transparent tubes or containers in which the culture is mixed by either a pump or air bubbling (Lee 1986; Chaumont 1993; Richmond 1990; Tredici 2004), tubular photobioreactors (For example, see Tamiya et al. (1953), Pirt et al. (1983), Gudin and Chaumont 1983, Chaumont et al. 1988; Richmond et al. 1993) and flat plate-type photobioreactors, such as those described in Samson and Leduy (1985), Ramos de Ortega and Roux (1986), Tredici et al. (1991, 1997) and Hu et al. (1996, 1998a,b).

As used herein, “conditions suitable to promote production” means that the conditions employed result in algal production of medium chain length fatty acids equal to at least 5% of total dry cell weight, and preferably 10%, 15%, 20%, 25%, or more.

The methods of the invention comprise extracting oil (ie: total fatty acids) from algae. Any suitable process for extracting oil from the algae can be used, including but not limited to solvent extraction and supercritical fluid extraction. Initially, algae are harvested from liquid culture in the photobioreactor using a suitable harvesting method (such as centrifugation, dissolved air floatation, membrane filtration, polymer-assisted flocculation, etc, singularly or in combination). The harvested algae can then be dried, if desired, using any suitable technique (such as sun-drying, drum-drying, freeze drying, or spray-drying) The resulting dried algae can be in any useful form, including but not limited to a form of algal flour.

As used herein, a “medium chain length fatty acid subset” is the medium chain length fatty acid produced by a given algal strain. Thus, culturing an algal strain that can produce large quantities of a medium chain length fatty acid subset under conditions suitable to promote production of the medium chain fatty acid subset, results in production of a medium chain length fatty acid subset that comprises at least 5% of total dry cell weight. The subset may comprise medium chain length fatty acids of any chain length or combination of chain lengths. The methods comprise use of a first algal strain that produces a first medium chain fatty acid subset and one or more further algal strains to produce a second or further medium chain fatty acid, where neither the oil from the first algal strain by itself nor the oil from any one of the one or more further algal strains by itself comprise detectable levels of each of carbon chain length C10, C12, and C14 fatty acids. Thus, where two algal strains are used, the methods comprise production of two medium chain fatty acid subsets (where neither algal strain individually produces a medium chain length fatty acid subset comprising C10, C12, and C14 fatty acids); where three algal strains are used the methods comprise production of three medium chain fatty acid subsets (where none of the three algal strains individually produce a medium chain length fatty acid subset comprising C10, C 12, and C14 fatty acids), and so on.

As used herein a “medium chain length combination” is a combined medium-chain length product (fatty acids or hydrocarbons) from the first algal strain and one or more other algal strains, where the medium chain length combination comprises carbon chain length C10, C12, and C14 fatty acids or hydrocarbons. The medium chain length combination may comprise either medium chain length fatty acids or medium chain length hydrocarbons, depending on the stage of processing the product is at. In one embodiment, the first algal strain and the one or more algal strains are co-cultured; in this case a medium chain length combination comprising medium chain length fatty acids is obtained upon oil extraction; if the medium chain length combination is then further processed to produce a hydrocarbon faction (see below), then the medium chain length combination will comprise medium chain length hydrocarbons after hydrocarbon fractionation. In another embodiment, the first algal strain and the one or more further algal strains are cultured separately; in this embodiment, the medium chain length combination is obtained sometime after oil extraction. For example, the first and second (or further) subsets can be combined immediately after oil extraction (resulting in a medium chain length combination comprising medium chain length fatty acids); or after other steps, such as after hydrocarbon fractionation, or after production of one or more fractions enriched in medium chain length hydrocarbons (see below), either of which results in a medium chain length combination comprising medium chain length hydrocarbons. As will be apparent to one of skill in the art, if three or more algal strains are used, they could all be co-cultured, or a subset could be co-cultured while other algal strains are cultured separately, and thus the combination of their medium chain length fatty acid subset or medium chain length hydrocarbons may comprise multiple combination events.

The medium chain length combination comprises carbon chain length C10, C12, and C14 fatty acids or hydrocarbons, wherein neither oil extracted from the first algal strain by itself, nor oil extracted from any one of the one or more further algal strains by itself comprises detectable levels of each of carbon chain length C10, C12, and C14 fatty acids.

The methods of this first aspect comprise the use of two or more algal strains where neither the oil from the first algal strain by itself nor the oil from the one or more further algal strains by themselves comprise detectable levels of each of carbon chain length C10, C12, and C14 fatty acids. As used herein, “detectable” levels mean that a given carbon chain length fatty acid represents at least 1% of the total fatty acid product in oil obtained from the algal strain.

As will be apparent to those of skill in the art, oil extraction from algae can be accompanied by extraction of other algal biomass that is separated from the oil during the extraction process. Thus, in another embodiment, the methods of the invention further comprise isolating algal biomass. Such biomass can include, but is not limited to, bulk products (useful, for example, for animal feed and biofertilizer); ethanol and methane (requires subsequent fermentation; useful, for example, in energy production); and specialty products, including but not limited to pigments (chlorophyll), polymers, carotenoids (e.g., beta-carotene, zeaxanthin, lutein, and astaxanthin), and polyunsaturated fatty acids.

In a further embodiment, the methods further comprise converting oil extracted from the first algal strain and the one or more further algal strains into a hydrocarbon fraction (ie: conversion of fatty acids into hydrocarbons). Any suitable process for converting algal fatty acids into hydrocarbons can be used, including but not limited to a deoxygenation/hydroxylation process, such as by chemical catalysis or hydrogen loading. A medium chain length combination prepared following hydrocarbon fractionation comprises medium chain length hydrocarbons. Such a medium chain length combination can be produced in whole or in part (by combination of hydrocarbon fractions produced from less than all of the algal strains employed) after hydrocarbon fractionation, or hydrocarbon fractionation can be performed separately on oil extracted from each algal strain. At least 30% of the hydrocarbons present in the hydrocarbon fraction are medium chain length hydrocarbons; in further embodiments, at least 35%, 40%, 45%, 50%, 55%, or more of the hydrocarbons present in the hydrocarbon fraction are medium chain length hydrocarbons.

As will be apparent to those of skill in the art, byproducts of hydrocarbon conversion, such as lighter fractions of hydrocarbons (e.g., C1-C6) and/or glycerol (glycerin), can also be obtained during hydrocarbon fractionation. Thus in a further embodiment, the methods further comprises isolating short-chain hydrocarbon molecules (C1-C6) and/or glycerol. The short chain hydrocarbons can be used, for example, to make tail gas or gasoline. Glycerol has many uses, including but not limited to use in pharmaceutical products (used as/in, for example, lubricant, humectant, expectorant, cough syrup, etc.), personal care products (used as/in, for example, emollient, lubricant, humectant, solvent, toothpaste, mouthwash, skin care products, soap, etc.) and food/beverage products (sweetener, filler, etc.).

In a further embodiment, the methods comprise refining the hydrocarbon fraction to produce one or more fractions enriched in medium chain length hydrocarbons, wherein the one or more fractions comprise one or more fractions enriched in carbon chain length C10, C12, and/or C14 hydrocarbons. For example, a separation/refining technology separates and concentrates desirable hydrocarbon fractions from a deoxygenation process, resulting in a series of refined fractions enriched with one or more hydrocarbons of specific carbon chain lengths. A medium chain length combination prepared following refining comprises medium chain length hydrocarbons. Such a medium chain length combination can be produced in whole or in part (by combination of hydrocarbons produced from less than all of the algal strains employed,) after refining, or refining can be performed separately on hydrocarbon fractions from each algal strain. The one or more fractions can comprise a single fraction that comprises C10, C12, and C14 chain length hydrocarbons, three separate fractions, one comprising C10 chain length hydrocarbons, one comprising C12 chain length hydrocarbons, and one comprising C14 chain length hydrocarbons, or other variations thereof. At least 90% of the hydrocarbons present in each fraction enriched in medium chain length hydrocarbons are of the desired chain length(s) hydrocarbon; in various further embodiments at least 91%, 92%, 93%, 94%, 95%, 96%, 97%, 98%, 99%, or more of the hydrocarbons present in each fraction enriched in medium chain length hydrocarbons are of the desired chain length(s) hydrocarbon.

Any suitable refining process can be used that serves to separate and concentrate fractions enriched in medium chain length fatty acids. In various embodiments, the refining comprises vacuum distillation or molecular distillation to separate and purify medium-chain (C8-C16) fatty acid (FA) or fatty acid methyl ester (FAME) from long-chain fatty acids (C18 or longer) or FAME. Vacuum distillation has been extensively used in petroleum refining, whereas molecular distillation is a newer technology that has been proved to be effective in separating one liquid from complex liquid mixtures. The vacuum distillation is similar in principle with the conventional fractional distillation (commonly called atmospheric distillation to distinguish it from the vacuum method), except that larger-diameter columns are used in vacuum distillation to maintain comparable vapor velocities at reduced operating pressures. A vacuum of 50 to 100 millimeters of mercury absolute is produced by a vacuum pump or steam ejector. The major advantage of vacuum distillation is that it allows for distilling heavier materials at lower temperatures than those that would be required at atmospheric pressure, thus avoiding thermal cracking of the components. An extension of the distillation process, superfractionation employs smaller-diameter columns with a much larger number of trays (100 or more) and reflux ratios exceeding 5:1. With such equipment it is possible to isolate a very narrow range of components or even pure compounds. Common applications involve the separation of high-purity solvents such as isoparaffins or of individual aromatic compounds for use as petrochemicals.

Molecular distillation is characterized by short exposure of the distilled liquid to elevated temperatures, high vacuum in the distillation space, and a small distance between the condenser and evaporator. The short residence of the liquid on the evaporating cylinder, in the order of a few seconds to 1 min, is guaranteed by distributing the liquid in the form of a uniform thin film. By reducing the pressure of non-condensable gas in the evaporator to lower than 0.1 Pa, a reduction in distillation temperatures can be obtained. Molecular distillation shows promise in the separation, purification and concentration of natural products, usually composed of complex and thermally sensitive molecules. Furthermore, this process has advantages over other techniques that use solvents as the separating agent, avoiding problems with toxicity. Centrifugal and falling films are two basic types of molecular distillation units that use short exposure of the distilled liquid to the evaporating cylinder. These types of distillation units have been used to demonstrate and compare the distillation of many different compounds, such as fatty acids, including the isomers with same carbon numbers in the molecular structures (for example: this technology can be used to separate C18: 3 from C18: 2, C18: 1 or C18: 0).

The refining process results in one or more refined oils enriched with one or more medium chain length fatty acids (for example, C10, C11, C12, C13, or C14). In a further embodiment the one or more fractions further comprise one or more fractions enriched in carbon chain length C16 fatty acids.

In another embodiment, the methods further comprise blending one or more of the medium chain length hydrocarbon fractions. Such blending can comprise any combination of medium chain length fatty acid fractions desired for a given purpose (ie: C10 and C12; C12 and C14; C10 and C14; C8, C10 and C16, etc.). For example, blending can result in a series of refined oils enriched with two or more hydrocarbons of specific carbon chain lengths.

In one embodiment, blending can be used to produce kerosene. As used herein, “kerosene” is a distribution of a variety of hydrocarbons in the C8-C16 range; preferably in the C10-C16, C8-C14, or C10-C14 range, and can be used, for example, in jet engine fuel (including but not limited to Jet-A, Jet-A1, Jet-B, JP-4, JP-5, JP-7, and JP-8); rocket fuel (including but not limited to RP-1); heating fuel (such as in kerosene heaters, portable stoves, and other heating sources); and to power appliances where electrical power is not otherwise available. It will be understood by those of skill in art that the kerosene can also be produced by appropriate production of medium chain length hydrocarbon fractions from the hydrocarbon fraction. In one embodiment, producing kerosene comprises combining two or more of the fractions enriched in medium chain hydrocarbons, where the resulting kerosene comprises at least 50% C10, C12, and C14 chain length hydrocarbons; in various further embodiments, at least 55%, 60%, 65%, 70%, 75%, 89%, 85%, 90%, 95%, 98% of carbon chain length C10, C12, and C14 hydrocarbons. The fractions so combined may comprise medium chain length hydrocarbons of the same type or different. In another embodiment, the kerosene may further comprise carbon chain length C16, C8 and/or C9 fatty acids each, if present, at 15% or less of the total hydrocarbon present in the kerosene; in preferred embodiments, each, if present, at less than 10%, 9%, 8%, 7%, 6%, 5%, 4%, 3%, 2% or less of the total hydrocarbon present in the kerosene.

Acceptable JP-8 surrogate fuel can thus be obtained by the blending of one or more fractions enriched in medium chain length hydrocarbons along with other additives according to the specification and qualification of petroleum derived JP-8 or other aviation fuels

In a further embodiment of all of the embodiments of the first aspect of the invention, the first algal strain and the one or more further algal strains are selected from the group consisting of Pinguiococcus pyrenoidosus, Aphanocapsa sp. (Kenyon, 1972), Biddulphia aurita (Orcutt & Pattersonl975), Crypthecodinium sp., Emiliania huxleyi (Volkman et al. 1981), Nitzschia alba (Tomabene et al. 1974), Pymnesium parvum (Lee & Loeblich 1971), Skeletonema costatum (Ackman et al. 1964), and Trichodesmium erythraeum (Parker et al. 1967). The types of medium chain fatty acids produced these organisms (and thus the potential medium chain fatty acid subsets) can be found in FIG. 1 or Table 1; based on the teachings herein, those of skill in the art will understand which algal strains to use, depending on the type of medium chain length combination desired. In specific embodiments, the algal strains are identified as follows:

| | |
|---|---|
| Pinguiococcus pyrenoidosus | CCMP 2078 |
| (Pinguiophyceae) | |
| Crypthecodinium sp | CCMP 316 |
| Aphanocapsa sp: | CCMP2524 |
| Odontella aurita: | CCMP145 |
| Emiliania huxleyi: | CCMP1742 |
| Nitzschia alba: | CCMP2426 |
| Prymnesium parvum: | CCMP1962 |
| Skeletonema costatum: | CCMP1281 |
| Trichodesmium sp: | CCMP1985 |

All of the algal strains can be obtained from CCMP address: Provasoli-Guillard National Center for the Culture of Marine Phytoplankton, Bigelow Laboratory for Ocean Sciences, P.O. Box 475, 180 McKown Point Road, West Boothloay Harbor, Me. 04575, U.S.A.)

In a second aspect, the present invention provides methods for producing algal medium chain length fatty acids, comprising

(a) culturing Pinguiococcus pyrenoidosus under conditions to promote production of medium chain length fatty acids; and

(b) extracting oil from the cultured Pinguiococcus pyrenoidosus wherein the extracted oil comprises C14 and C16 chain length fatty acids.

The inventors have discovered that Pinguiococcus pyrenoidosus, such as variant CCMP 2078 (described below), are capable of producing large amounts of medium chain length fatty acids. Thus, the methods of this second aspect of the invention can be used for various purposes, including but not limited to production of algal-based kerosene substitutes, high quality detergents, and research reagents (for example, isolated hydrocarbon fractions of a single chain length for use as standards that can be optionally labeled for research use).

Terms used in this second aspect of the invention have the same meanings as provided in the first aspect of the invention, and embodiments of the first aspect are also applicable to this second aspect. In a further embodiment, the methods comprise converting oil extracted from Pinguiococcus pyrenoidosus into a hydrocarbon fraction, where hydrocarbon fraction is as defined above. In another embodiment, the methods further comprise refining the hydrocarbon fraction to produce one or more fractions enriched in medium chain length hydrocarbons, wherein the one or more fractions comprises at least one fraction enriched in carbon chain length C14 hydrocarbons. In a further embodiment, the one or more fractions comprise at least one fraction enriched in carbon chain length C16 hydrocarbons. In a further embodiment, the method further comprises blending the one or more fractions enriched in medium chain length hydrocarbons to produce, for example, kerosene. Such blending may further comprise blending with medium chain length hydrocarbon fractions derived from another algal strain, such as C10 and/or C12 chain length hydrocarbon chains (for example, those derived from Crypthecodinium sp. and/or Trichodesmium erythraeum). The methods of this second aspect may also comprise isolating algal biomass, and/or isolating short-chain hydrocarbon molecules and/or glycerol, as disclosed above in the first aspect of the invention.

In a third aspect, the present invention provides methods for producing algal medium chain length fatty acids or hydrocarbons, comprising

(a) culturing Pinguiococcus pyrenoidosus under conditions to promote production of medium chain length fatty acids;

(b) culturing one or more further algal strains that can produce and accumulate large quantities of C10 and/or C12 chain length fatty acids, wherein the culturing is conducted under conditions suitable to promote production of the C10 and/or C12 chain length fatty acids; and

(c) extracting oil from the cultured Pinguiococcus pyrenoidosus and the one or more further algal strains to produce a medium chain length combination; wherein the medium chain length combination comprises carbon chain length C14 and one or more of carbon chain length C10 and C12 fatty acids or hydrocarbons.

The methods of this third aspect of the invention can be used for various purposes, including but not limited to production of algal-based kerosene substitutes, high quality detergents, and research reagents (for example, isolated hydrocarbon fractions of a single chain length for use as standards that can be optionally labeled for research use). Terms used in this third aspect of the invention have the same meanings as provided in the first aspect of the invention, and embodiments of the first aspect are also applicable to this third aspect. In various embodiments, the one or more further algal strains are one or both of Crypthecodinium sp. and Trichodesmium erythraeum. In a further embodiment, the medium chain length combination comprises carbon chain length C10, C12, and C14 fatty acids or hydrocarbons. In a further embodiment, the medium chain length combination comprises carbon chain length C16 fatty acids or hydrocarbons. In a further embodiment, the medium chain length combination is prepared by combining oil extracted from the Pinguiococcus pyrenoidosus and the one or more further algal strains after oil extraction. In a further embodiment, the medium chain length combination is prepared by extracting oil from a culture comprising both the Pinguiococcus pyrenoidosus and the one or more further algal strains. In a further embodiment, the methods comprise converting oil extracted from Pinguiococcus pyrenoidosus and the one or more further algal strains into a hydrocarbon fraction, where hydrocarbon fraction is as defined above. In another embodiment, the methods further comprise refining the hydrocarbon fraction to produce one or more fractions enriched in medium chain length hydrocarbons, wherein the one or more fractions comprises one or more fractions enriched in carbon chain length C10, C12, and/or C14 hydrocarbons. In a further embodiment, the one or more fractions comprise at least one fraction enriched in carbon chain length C16 hydrocarbons. In a further embodiment, the method further comprises blending one or more of the fractions enriched in medium chain length hydrocarbons to, for example, produce kerosene. The methods of this third aspect may also comprise isolating algal biomass, and/or isolating short-chain hydrocarbon molecules and/or glycerol, as described above. In a further embodiment of any of the above, the one or more further algal strains comprises a second algal strain and a third algal strain, wherein the third algal strain is selected from the group consisting of Aphanocapsa sp., Biddulphia aurita, Crypthecodinium sp., Emiliania huxleyi, Nitzschia alba, Prymnesium parvum, Skeletonema costatum, and Trichodesmium erythraeum.

In a fourth aspect, the present invention provides methods for producing algal medium chain length fatty acids or hydrocarbons, comprising

(a) culturing Trichodesmium erythraeum under conditions to promote production of medium chain length fatty acids, wherein the medium chain length fatty acids comprise C10 chain length fatty acids;

(b) culturing Crypthecodinium sp. under conditions to promote production of medium chain length fatty acids, wherein the medium chain length fatty acids comprise C12 chain length fatty acids; and

(c) extracting oil from the cultured Trichodesmium erythraeum and the Crypthecodinium sp. to produce a medium chain length combination; wherein the medium chain length combination comprises carbon chain length C10 and C12 fatty acids or hydrocarbons.

The methods of this fourth aspect of the invention can be used for various purposes, including but not limited to production of algal-based kerosene substitutes, high quality detergents, and research reagents (for example, isolated hydrocarbon fractions of a single chain length for use as standards that can be optionally labeled for research use). Terms used in this fourth aspect of the invention have the same meanings as provided in the first aspect of the invention, and embodiments of the first aspect are also applicable to this fourth aspect. In one embodiment, the medium chain length combination further comprises carbon chain length C14 fatty acids or hydrocarbons. In a further embodiment, the methods further comprise (d) culturing one or more algal strains selected from the group consisting of Pinguiococcus pyrenoidosus, Aphanocapsa sp., Biddulphia aurita, Emiliania huxleyi, Nitzschia alba, Prymnesium parvum, and Skeletonema costatum under conditions to promote production of medium chain length fatty acids, wherein the medium chain length fatty acids comprise C14 and/or C16 chain length fatty acids; and (e) extracting oil from the cultured one or more algal strains to be included in the medium chain length combination; and wherein the medium chain length combination comprises carbon chain length C14 and/or C16 fatty acids or hydrocarbons. In a further embodiment, the medium chain length combination is prepared by combining oil extracted from the culture Trichodesmium erythraeum and Crypthecodinium sp. after oil extraction. In another embodiment, the medium chain length combination is prepared by extracting oil from a culture comprising both the Trichodesmium erythraeum and Crypthecodinium sp. In another embodiment, the medium chain length combination is prepared by combining oil extracted from the culture Trichodesmium erythraeum, Crypthecodinium sp., and the one or more algal strains after oil extraction. In a further embodiment, the medium chain length combination is prepared by extracting oil from a culture comprising the Trichodesmium erythraeum, the Crypthecodinium sp., and the one or more algal strains. In a further embodiment, the methods further comprise converting the oil extracted from the algal strains into a hydrocarbon fraction, as defined above. The methods may further comprise refining the hydrocarbon fraction to produce one or more fractions enriched in medium chain length hydrocarbons, wherein the one or more fractions comprises one or more fractions enriched in carbon chain length C10 and C12 hydrocarbons, and optionally C14 and/or C16 hydrocarbons. The methods may further comprise blending one or more of the fractions enriched in medium chain length hydrocarbons to, for example, produce kerosene. In various further embodiments, the methods further comprise isolating algal biomass, and/or isolating short-chain hydrocarbon molecules and/or glycerol, as discussed in detail in the first aspect of the invention.

In a fifth aspect, the present invention provides a composition comprising two or more isolated algal strains selected from the group consisting of Pinguiococcus pyrenoidosus, Aphanocapsa sp., Biddulphia aurita, Crypthecodinium sp., Emiliania huxleyi, Nitzschia alba, Prymnesium parvum, Skeletonema costatum, and Trichodesmium erythraeum, wherein the two or more algal strains make up at least 90% of the algae present in the composition. In further embodiments, at least 95%, 98%, or 99% of the algae present in the composition are of the recited algal type. The isolated algal composition can be cultured or stored in solution, frozen, dried, or on solid agar plates. Alternatively, the compositions may comprise harvested algal compositions (wet or dried) in, for example, the form of an algal flour. In specific embodiments, the algal strains are identified as follows:

| | |
|---|---|
| Pinguiococcus pyrenoidosus | CCMP 2078 |
| (Pinguiophyceae) | |
| Crypthecodinium sp | CCMP 316 |
| Aphanocapsa sp: | CCMP2524 |
| Odontella aurita: | CCMP145 |
| Emiliania huxleyi: | CCMP1742 |
| Nitzschia alba: | CCMP2426 |
| Prymnesium parvum: | CCMP1962 |
| Skeletonema costatum: | CCMP1281 |
| Trichodesmium sp: | CCMP1985 |

All of the algal strains can be obtained from CCMP address: Provasoli-Guillard National Center for the Culture of Marine Phytoplankton, Bigelow Laboratory for Ocean Sciences, P.O. Box 475, 180 McKown Point Road, West Boothloay Harbor, Me. 04575, U.S.A.)

The compositions of this aspect of the invention can be used, for example, in the methods of the invention. In one embodiment, the composition comprises three or more isolated algal species selected from the group. In a further embodiment, the two or more isolated algal strains comprise Pinguiococcus pyrenoidosus. In a still further embodiment, the two or more isolated algal strains comprise one or both of Crypthecodinium sp. and Trichodesmium erythraeum.

In a sixth aspect, the present invention provides a substantially pure culture comprising
(a) growth medium; and
(b) the composition of any embodiment of the compositions of the fifth aspect of the invention.

As used herein, the term “growth medium” refers to any suitable medium for cultivating algae of the present invention. The algae of the invention can grow photosynthetically on CO₂ and sunlight, plus a minimum amount of trace nutrients. The volume of growth medium can be any volume suitable for cultivation of the algae for any purpose, whether for standard laboratory cultivation, to large scale cultivation for use in, for example, medium chain fatty acid production. Suitable algal growth medium can be any such medium, including but not limited to BG-11 growth medium (see, for example, Rippka, 1979); culturing temperatures of between 10° and 38° C. are used; in other embodiments, temperature ranges between 15° and 30° are used. Similarly, light intensity between 20 μmol m⁻²s⁻¹ to 1000 μmol m⁻²s⁻¹ is used; in various embodiments, the range may be 100 μmol m⁻²s⁻¹ to 500 μmol m⁻²s⁻¹ or 150 μmol m⁻²s⁻¹ to 250 μmol m⁻²s⁻¹. Further, aeration is carried out with between 0% and 20% CO₂; in various embodiments, aeration is carried out with between 0.5% and 10% CO₂, 0.5% to 5% CO₂, or 0.5% and 2% CO₂.

For maintenance and storage purposes, the compositions of the invention may be maintained in standard artificial growth medium. For regular maintenance purposes, the compositions can be kept in liquid cultures or solid agar plates under either continuous illumination or a light/dark cycle of moderate ranges of light intensities (10 to 40 μmol m⁻² s⁻¹) and temperatures (18° C. to 25° C.). The culture pH may vary from pH 6.5 to pH 9.5. No CO₂ enrichment is required for maintenance of the compositions. In various non-limiting examples, the temperature of culture medium in growth tanks is preferably maintained at from about 10° C. to about 38° C., in further embodiments, between about 20° C. to about 30° C. In various embodiments, the growth medium useful for culturing the compositions of the present invention comprises wastewater or waste gases, as discussed above.

In a seventh aspect, the present invention provides an algal-derived hydrocarbon fraction. In one embodiment, the algal-derived hydrocarbon fraction is produced by the methods of any embodiment of any one of the first, second, third, or fourth aspects of the invention. Terms and embodiments of the first, second, third, and fourth embodiments are applicable to this seventh embodiment. At least 30% of the hydrocarbons present in the hydrocarbon fraction are medium chain length hydrocarbons; in further embodiments, at least 35%, 40%, 45%, 50%, 55%, or more of the hydrocarbons present in the hydrocarbon fraction are medium chain length hydrocarbons.

In an eighth aspect, the present invention provides an algal-derived, isolated medium chain hydrocarbon fraction. In one embodiment, algal-derived, isolated medium chain hydrocarbon fraction is produced by the methods of any embodiment of any one of the first, second, third, or fourth aspects of the invention. Terms and embodiments of the first, second, third, and fourth embodiments are applicable to this seventh embodiment. At least 90% of the hydrocarbons present in each fraction enriched in medium chain length hydrocarbons are of the desired chain length(s) hydrocarbon; in various further embodiments at least 91%, 92%, 93%, 94%, 95%, 96%, 97%, 98%, 99%, or more of the hydrocarbons present in each fraction enriched in medium chain length hydrocarbons are of the desired chain length(s) hydrocarbon.

In a ninth aspect, the present invention provides algal-derived kerosene. In one embodiment, algal-derived kerosene is produced by the methods of any embodiment of any one of the first, second, third, or fourth aspects of the invention. Terms and embodiments of the first, second, third, and fourth embodiments are applicable to this seventh embodiment. In one embodiment, producing kerosene comprises combining two or more of the fractions enriched in medium chain hydrocarbons, where the resulting kerosene comprises at least 50% C10, C12, and C14 chain length hydrocarbons; in various further embodiments, at least 55%, 60%, 65%, 70%, 75%, 89%, 85%, 90%, 95%, 98% of carbon chain length C10, C12, and C14 hydrocarbons. The fractions so combined may comprise medium chain length hydrocarbons of the same type or different. In another embodiment, the kerosene may further comprise carbon chain length C16, C8 and/or C9 fatty acids each, if present, at 15% or less of the total hydrocarbon present in the kerosene; in preferred embodiments, each, if present, at less than 10%, 9%, 8%, 7%, 6%, 5%, 4%, 3%, 2% or less of the total hydrocarbon present in the kerosene.

### EXAMPLE 1

A general process diagram of the proposed algae-based jet fuel production technology is shown in FIG. 2.

In various non-limiting examples, the following processes can be carried out in conjunction with algae-based medium chain length fatty acid production:
Production of algal feedstock using a number of selected algal species grown in one or more photobioreactors of same or different designs. Each selected algal species will produce large quantities of oil enriched with one or more medium-chain length fatty acids/esters.
Oil-rich cells are harvested and dried in a form of algal flour.
Algal flour is subjected to solvent extraction using a chemical extraction method. A supercritical liquid extraction method can also be employed as an alternative.
Resulting algal oil is subjected to a deoxygenating/hydroxylation process to convert algal oil to hydrocarbons.
A separation/refining technology separates and concentrates desirable hydrocarbon fractions from the deoxygenation process. As a result, a series of refined oils enriched with one or more hydrocarbons of specific carbon chain lengths will be produced.
Acceptable JP-8 surrogate fuel is obtained by the blending of several refined algal oils along with other additives according to the specification and qualification of petroleum derived JP-8 or other aviation fuels.
As a by-product from algal oil extraction, algal biomass residues are prepared and used as bulk material in, for example, protein-rich animal feed or polysaccharide-rich biopolymers and fertilizer. Some specialty products such as high-value carotenoids (e.g., beta-carotene, zeaxanthin, lutein, and astaxanthin) can also be extracted and separated from selected algal strains.
High carbohydrate-containing biomass residues from oil extraction process can also be obtained and used a substrate for fermentation or anaerobic digestion to produce ethanol and/or methane, which in turn can be used to generate electricity/energy necessary for algal mass culture and oil processing/refinery processes. Remaining undigested biomass residues can be incinerated for additional heat and electricity. The generation of CO₂ from anaerobic digestion and incineration processes can be recycled back into the photobioreactor to be used by the algae, resulting in zero net CO₂ emissions.
The methods of the invention employ algae for medium chain fatty acid extraction and conversion into hydrocarbons, thus minimizing or eliminating the need to use cracking for hydrocarbon production, thus greatly reducing costs and energy consumption. Furthermore, resulting short-chain hydrocarbon molecules can be isolated as by-products of the methods to make tail gas or gasoline.

### EXAMPLE 2

We have performed screening for medium-chain oil-producers from numerous algal species/strains isolated by and maintained in our lab. One of the algal strains tested in our lab is a marine alga Pinguiococcus pyrenoidosus (Pinguiophyceae) CCMP 2078 (Provasoli-Guillard National Center for the Culture of Marine Phytoplankton, Bigelow Laboratory for Ocean Sciences, P.O. Box 475, 180 McKown Point Road, West Boothloay Harbor, Me. 04575, U.S.A.), which has the ability to produce lipids enriched with C14 fatty acid, which can make up 30 to 50% of total fatty acids produced in the cell. The fatty acid composition of Pinguiococcus pyrenoidosus is disclosed in Table 1.

TABLE 1
Fatty acid composition of Pinguiococcus pyrenoidosus.

| Fatty acids | % of total fatty acids |
|---|---|
| 14:0 | 49.42 |
| 16:0 | 30.15 |
| 16:1 | 1.02 |
| 18:0 | 2.13 |
| 18:1 | 3.8 |
| 18:2 | 1.62 |

The alga was grown in h/2 growth medium and exposed to a light intensity of 200 μmol m⁻² s⁻¹ and at 25° C.

FIG. 1 lists eight (8) medium-chain oil-producing algal species as examples that contain medium-chain fatty acids as the dominant carbon chain length (30 to 70% of total fatty acids).

Our investigations have revealed that Crypthecodinium sp CCMP 316 (Provasoli-Guillard National Center for the Culture of Marine Phytoplankton, Bigelow Laboratory for Ocean Sciences, P.O. Box 475, 180 McKown Point Road, West Boothloay Harbor, Me. 04575, U.S.A.). exhibits a growth rate with average doubling times ranging from 5 to 10 hours, comparable to many rapid-growing algae used for commercial production. The content of C12+C14 fatty acids of this organism can make up over 40% of total cell dry weight. This strain was also found to be able to undergo heterotrophic growth using glucose as a sole carbon and energy source, making it particularly suitable for outdoor mass culture where the cell produces organic compounds through photosynthesis during the day, while continuing biomass/oil production in the presence of glucose in the night. Furthermore, this strain can accumulate C12 and C14 fatty acids under normal growing conditions, indicative of their constitutive expression of the genes/enzymes involved in lipid biosynthetic pathways, a desirable metabolic feature that will ensure concomitant maximum sustainable production of cell mass and C12 and C14 fatty acids under optimal culture conditions. This is in great contrast to many previously reported algal species/strains which accumulate long-chain (C16 and C18) fatty acids only under adverse growth conditions, resulting in reduced biomass productivity. Our C10 to C14 algal strains are also in contrast to the colonial green alga Botryococcous braunii, which grows extremely slowly (e.g., 1/10 the rate of a unicellular Chlorella) and is able to produce only long-chain hydrocarbons (C23 to C40) under environmental stress conditions, which by themselves cannot be readily used as kerosene-based JP-8, but have to be subjected to thermo/chemical cracking, an energy intensive process.

### REFERENCES

1. Kenyon, C. N. 1972. Fatty acid composition of unicellular strains of blue-green algae. J. Bacteriol. 109: 827-834.
2. Orcutt, D. M. and Patterson, G. W. 1975. Sterol, fatty acid and elemental composition of diatoms grown in chemically media. Comp. Biochem. Physiol. 50B: 579-583.
3. Volkman, J. K., Smith, D. J., Eglinton, G., Forsberg, T. E. V. and Corner, E. D. S. 1981. Sterol and fatty acid composition of four marine haptophycean algae. J. Mar. Biol. Ass. U. K. 61: 509-527.
4. Tornabene, T. G., Kates, M. and Volcanl, B. E. 1974. Sterol aliphatic hydrocarbons, and fatty acids of a nonphotosynthetic diatom, Nitzschia alba. Lipids. 9: 279-284.
5. Lee, R. F. and Loeblich III, A. R. 1971. Distribution of 21:6 hydrocarbon and its relationship to 22:6 fatty acid in algae. Phytochemistry. 10: 593-602.
6. Ackman, R. G., Jangaard, P. M., Hoyle, R. J. and Brockerhoff, H. 1964. Origin of marine fatty acids. I. Analyses of the fatty acids produced by the diatom Skeletonema costatum. J. Fish. Res. Bd. Can. 21: 747-756.
7. Parker, P. L., van Baalen, C. and Maurer, L. 1967. Fatty acids in eleven species of bluegreen algae: geochemical significance. Science. 155: 707-708.
8. Shay, E. G. 1993. Diesel fuel from vegetable oils: Status and opportunities. Biomass and Bioenergy 4(4): 227-242.
9. Srivastava, A. and Prasad, R. 2000. Triglycerides-based diesel fuels. Renewable & Sustainable Energy reviews. 4: 111-133.

1. A method for producing algal medium chain length fatty acids or hydrocarbons, comprising:
(a) culturing a first algal strain selected from the group consisting of Pinguiococcus pyrenoidosus, Aphanocapsa sp., Biddulphia aurita, Crypthecodinium sp., Emiliania huxleyi, Nitzschia alba, Prymnesium parvum, Skeletonema costatum, and Trichodesmium erythraeum wherein said first algal strain produces a first medium chain length fatty acid subset wherein at least 20% of the fatty acids in said subset are medium chain length fatty acids, wherein the culturing is conducted under conditions suitable to promote production of the first medium chain fatty acid subset;
(b) culturing one or more further algal strains that produce a second medium chain length fatty acid subset wherein at least 20% of the fatty acids in said subset are medium chain length fatty acids, wherein the culturing is conducted under conditions suitable to promote production of the second medium chain fatty acid subset; and
(c) extracting oil from the first algal strain and the one or more further algal strains to produce a medium chain length combination; wherein the medium chain length combination comprises carbon chain length C10, C12, and C14 fatty acids or hydrocarbons, and wherein neither the oil from the first algal strain by itself nor the oil from any one of the one or more further algal strains by itself comprise detectable levels of each of carbon chain length C10, C12, and C14 fatty acids;
said method optionally further comprising converting oil extracted from the first algal strain and the one or more further algal strains into a hydrocarbon fraction and refining the hydrocarbon fraction to produce one or more fractions enriched in medium chain length hydrocarbons, wherein the one or more fractions comprises one or more fractions enriched in carbon chain length C10, C12, and C14 hydrocarbons.

2. The method of claim 1, wherein the medium chain length combination further comprises carbon chain length C16 fatty acids or hydrocarbons.

3. The method of claim 1, wherein the first algal strain and the one or more further algal strains are cultured as separate cultures or are cultured as a co-culture.

4-6. (canceled)

7. The method of claim 1, wherein the one or more fractions further comprises one or more fractions enriched in carbon chain length C16 hydrocarbons.

8. The method of claim 1, further comprising producing kerosene from the one or more fractions enriched in medium chain length hydrocarbons.

9. The method of claim 1, further comprising isolating:
a) an algal biomass residue and/or
b) short-chain hydrocarbon molecules and/or glycerol.

10-11. (canceled)

12. The method of claim 1, wherein the one or more further algal strains comprises at least a second algal strain and a third algal strain that is different from said first algal strain and independently is selected from the group consisting of Pinguiococcus pyrenoidosus, Aphanocapsa sp., Biddulphia aurita, Crypthecodinium sp., Emiliania huxleyi, Nitzschia alba, Prymnesium parvum, Skeletonema costatum, and Trichodesmium erythraeum.

13. (canceled)

14. A method for producing algal medium chain length fatty acids, comprising
(a) culturing Pinguiococcus pyrenoidosus under conditions suitable to promote production of medium chain length fatty acids; and
(b) extracting oil from the cultured Pinguiococcus pyrenoidosus wherein the extracted oil comprises C14 and C16 chain length fatty acids.

15. The method of claim 14 further comprising converting oil extracted from Pinguiococcus pyrenoidosus into a hydrocarbon fraction, and optionally refining the hydrocarbon fraction to produce one or more fractions enriched in medium chain length hydrocarbons, wherein the one or more fractions comprises at least one fraction enriched in carbon chain length C14 hydrocarbons.

16-20. (canceled)

21. A method for producing algal medium chain length fatty acids or hydrocarbons, comprising
(a) culturing Pinguiococcus pyrenoidosus under conditions suitable to promote production of medium chain length fatty acids;
(b) culturing one or more further algal strains that produce and accumulate large quantities of C10 and/or C12 chain length fatty acids, wherein the culturing is conducted under conditions suitable to promote production of the C10 and/or C12 chain length fatty acids; and
(c) extracting oil from the cultured Pinguiococcus pyrenoidosus and the one or more further algal strains to produce a medium chain length combination; wherein the medium chain length combination comprises carbon chain length C14 and one or more of C10 and C12 fatty acids or hydrocarbons
said method optionally further comprising converting the medium chain length combination into a hydrocarbon fraction and further comprising refining the hydrocarbon fraction to produce one or more fractions enriched in medium chain length hydrocarbons, wherein the one or more fractions comprises one or more fractions enriched in carbon chain length C10, C12, and C14 hydrocarbons.

22. (canceled)

23. The method of claim 21, wherein the medium chain length combination is prepared by combining oil extracted from the Pinguiococcus pyrenoidosus and the one or more further algal strains after oil extraction or by extracting oil from a culture comprising both the Pinguiococcus pyrenoidosus and the one or more further algal strains.

24-30. (canceled)

31. The method of claim 21, wherein the one or more further algal strains comprises a second algal strain and a third algal strain, wherein the third algal strain is selected from the group consisting of Aphanocapsa sp., Biddulphia aurita, Crypthecodinium sp., Emiliania huxleyi, Nitzschia alba, Prymnesium parvum, Skeletonema costatum, and Trichodesmium erythraeum.

32. A method for producing algal medium chain length fatty acids or hydrocarbons, comprising
(a) culturing Trichodesmium erythraeum under conditions suitable to promote production of medium chain length fatty acids, wherein the medium chain length fatty acids comprise C10 chain length fatty acids;
(b) culturing Crypthecodinium sp. under conditions suitable to promote production of medium chain length fatty acids, wherein the medium chain length fatty acids comprise C12 chain length fatty acids; and
(c) extracting oil from the cultured Trichodesmium erythraeum and the Crypthecodinium sp. to produce a medium chain length combination; wherein the medium chain length combination comprises carbon chain length C10 and C12 fatty acids or hydrocarbons
and optionally, wherein the medium chain length combination further comprises carbon chain length C14 fatty acids or hydrocarbons.

33. (canceled)

34. The method of claim 32, further comprising
(d) culturing one or more algal strains selected from the group consisting of Pinguiococcus pyrenoidosus, Aphanocapsa sp., Biddulphia aurita, Emiliania huxleyi, Nitzschia alba, Prymnesium parvum, and Skeletonema costatum under conditions suitable to promote production of medium chain length fatty acids, wherein the medium chain length fatty acids comprise C14 and/or C16 chain length fatty acids; and
(e) extracting oil from the cultured one or more algal strains to be included in the medium chain length combination; and wherein the medium chain length combination comprises carbon chain length C14 and/or C16 fatty acids or hydrocarbons
said method optionally further comprising converting the medium chain length combination into a hydrocarbon fraction and refining the hydrocarbon fraction to produce one or more fractions enriched in medium chain length hydrocarbons, wherein the one or more fractions comprises one or more fractions enriched in carbon chain length C10, C12, and C14 hydrocarbons.

35. The method of claim 32, wherein the medium chain length combination is prepared by combining oil extracted from the culture Trichodesmium erythraeum and Crypthecodinium sp. after oil extraction or wherein wherein the medium chain length combination is prepared by extracting oil from a co-culture comprising both the Trichodesmium erythraeum and Crypthecodinium sp.

36-42. (canceled)

43. A composition comprising two or more isolated algal strains selected from the group consisting of Pinguiococcus pyrenoidosus, Aphanocapsa sp., Biddulphia aurita, Crypthecodinium sp., Emiliania huxleyi, Nitzschia alba, Prymnesium parvum, Skeletonema costatum, and Trichodesmium erythraeum, wherein the two or more algal strains make up at least 90% of the algae present in the composition.

44-46. (canceled)

47. The composition of claim 43 wherein the two or more isolated algal strains comprise one or both of Crypthecodinium sp. and Trichodesmium erythraeum.

48. The composition of claim 47, wherein the two or more isolated algal strains further comprise an algal strain selected from the group consisting of Pinguiococcus pyrenoidosus, Aphanocapsa sp., Biddulphia aurita, Emiliania huxleyi, Nitzschia alba, Prymnesium parvum, and Skeletonema costatum.

49. A substantially pure culture comprising
(a) growth medium; and
(b) the composition of claim 43.

50. (canceled)

51. An isolated hydrocarbon fraction produced by the method of claim 7.

52. Kerosene produced by the method of claim 8.
